(19) United States
(12) Patent Application Publication
(10) Pub. No.: US 2003/0077811 A1
(43) Pub. Date: Apr. 24, 2003

(54) ADAMTS nucleic acids and proteins

(76) Inventors: Suneel S. Apte, Shaker Heights, OH (US); Satoshi Hirohata, Okayama (JP); David R. Eyre, Seattle, WA (US); Russell J. Fernandes, Redmond, WA (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(21) Appl. No.: 10/164,890

(22) Filed: Jun. 6, 2002

Related U.S. Application Data
(60) Provisional application No. 60/296,384, filed on Jun. 6, 2001.

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

The present invention is directed to ADAMTS3 and the corresponding protein ADAMTS-3 as well as variants, homologs, and equivalents, and their use in procollagen processing.

### CROSS REFERENCES TO RELATED APPLICATIONS

This application claims benefit of U.S. Provisional Application No. 60/296,384, filed Jun. 6, 2001.

### BACKGROUND OF THE INVENTION

The present invention relates generally to isolated nucleic acid molecules encoding proteins, and the proteins themselves, belonging to a subfamily of zinc metalloproteases referred to as “ADAMTS”, an abbreviation for A Disintegrin-like And Metalloprotease domain with ThromboSpondin type I motifs. Proteins in the ADAMTS subfamily all possess a Zn protease catalytic site consensus sequence (HEXXH+H), which suggests an intact catalytic activity for each of these proteins. The ADAMTS proteins also have putative N-terminal signal peptides and lack transmembrane domains, which suggests that the proteins in this subfamily are secreted. The proteins in the ADAMTS subfamily also possess at least one thrombospondin type (TSP 1) motif, which suggests a binding of these proteins to components of the extracellular matrix (ECM) or to cell surface components.

U.S. Pat. No. 6,391,610, which is incorporated herein in its entirety by reference thereto, describes certain members of the ADAMTS family member and their implications in a variety of diseases. It is believed that members of the ADAMTS family play a role in the cleavage of proteoglycan core proteins found in the extracellular matrix (e.g. versican, brevican, neuracan, and aggrecan, as well as collagen). It is expected that other members of the ADAMTS subfamily play a role in embryogenesis, implantation of a fertilized egg, angiogenesis, arthritic degradation of cartilage, inflammation, nerve regeneration, tumor growth, and metastases.

Collagens comprise the major structural proteins of the extracellular matrix (ECM) and exist in both fibril-forms (e.g., collagens I, II, III, V and XI) and nonfibrillar forms. Molecules belonging to both categories are homo-trimeric (e.g., collagen II) or hetero-trimeric (e.g., collagen I) assemblies of specific a-chains, each the product of a single gene. The molecular forms of collagens, as well as the specific supramolecular aggregates and assemblies they form, are often tissue specific, and provide specialized functions. For example, collagen I, the principal collagen of skin, is arranged in randomly oriented bundles which form a sheet in the dennis, but is arranged in parallel bundles in tendons. This reflects the different mechanical demands on these two tissues. Collagen II, a specific component of cartilage ECM, is arranged in an open, intercrossing pattern which traps glycosaminoglycans and facilitates resistance to compression.

The synthesis, secretion and assembly of collagens into specific supramolecular aggregates is a complex, multi-step process. Fibrillar collagens I, II and III are secreted as soluble procollagens comprising a long, continuous triple helical “collagenous” region with smaller polypeptide extensions (propeptides) at their amino (N) and carboxyl (C) ends. The removal of the propeptides by specific enzymes, the N- and C-propeptidases (proteinases), is a prerequisite for the correct assembly of collagens I and II into fibrils and for fibril growth. The procollagen C-propeptidase is identical to bone morphogenetic protein-1 (BMP-1) and processes all three of these fibrillar collagens. Biochemically distinct N-propeptidases with specificity for procollagen I and procollagen II or for procollagen III are known. One bovine and human procollagen I/II N-propeptidases has been cloned. It is known as ADAMTS-2.

The ADAMTS-2 protein (EC 3.4.24.14), also known as procollagen I/II amino-propetide processing enzyme or PCINP, catalyzes cleavage of native triple-helical procollagen I and procollagen II. The ADAMTS-2 protein also has an affinity for collagen XIV. Lack of the ADAMTS-2 protein is known to cause dermatosparaxis in cattle, or Ehlers-Danlos syndrome type VIIC (EDS-VIIC) in humans. EDS-VIIC is characterized clinically by severe skin fragility, and biochemically by the presence in skin of procollagen which is incompletely processed at the amino terminus. The molecular hallmark is the presence of irregular, thin, branched collagen fibrils in the dermis which appear “hieroglyphic” in cross section and contain procollagen I with an intact N-propeptide, termed pN-collagen I. Similar findings have been described very recently in Adamts2 transgenic knockout mice. Thus, it is believed that the ADAMTS-2 protein plays a role in processing of procollagen to mature collagen, an essential step for correct assembly of collagen into collagen fibrils.

A number of diseases have been shown to be caused by: synthesis of insufficient amounts of collagen; synthesis of defective collagen; or over-production of normal collagen in a form referred to as either fibrotic tissue or scars.

### SUMMARY OF THE INVENTION

As used herein, the following abbreviations have the following meanings:

“a.a.” is used herein to mean amino acids;

“EDS” is used herein to mean Ehlers-Danlos syndrome;

“BMP-1” is used herin to mean bone morphogenetic protein-1;

“PCR” is used herein to mean polymerase chain reaction;

“RT-PCR” is used herein to mean reverse transcriptase-PCR;

“kbp” is used herein to mean kilobase pairs;

“bp” is used herein to mean base pairs;

“nt” is used herein to mean nucleotides;

“ECM” is used herein to mean extracellular matrix;

“TS” is used herein to mean thrombospondin;

“GAPDH” is used herein to mean Glyceraldehyde 3-phosphate dehydrogenase;

“UV” is used herein to mean ultraviolet.

Where appropriate, approved nomenclature is used for human and mouse genes. ADAMTS2 and ADAMTS3 are human genes. Adamts2 and Adamts3 are the corresponding mouse orthologs. The protein products of the respective genes are designated ADAMTS-2 and ADAMTS-3. Trivial names for the protein products are procollagen N-propeptidase 1 (PCNP1) and procollagen N-propeptidase 2 (PCNP2).

The preferred nucleic acids of the invention are homologs and alleles of the nucleic acids of ADAMTS3. The invention further embraces functional equivalents, variants, analogs and fragments of the foregoing nucleic acids and also embraces proteins and peptides coded for by any of the foregoing. For a discussion of what is meant by “variant” and other defined terms, reference is made to U.S. Pat. No. 6,391,610, which is hereby incorporated in its entirety by reference thereto. The present invention also provides isolated polynucleotides which encode an ADAMTS-3 protein or a variant thereof, polynucleotide sequences complementary to such polynucleotides, vectors containing such polynucleotides, and host cells transformed or transfected with such vectors. The present invention also relates to antibodies which are immunospecific for one or more of the ADAMTS-3 proteins.

According to one aspect of the invention, an isolated nucleic acid molecule is provided. The invention further embraces nucleic acid molecules that differ from the foregoing isolated nucleic acid molecules in codon sequence due to the degeneracy of the genetic code. The invention also embraces complements of the foregoing nucleic acids.

Preferred isolated nucleic acid molecules are those comprising mammallian cDNAs or gene corresponding to ADAMTS3. Even more preferably the present invention relates to isolated nucleic acid molecules comprising human cDNA or genes corresponding to ADAMTS-3.

According to another aspect of the invention, isolated polypeptides (e.g. ADAMTS-3) coded for by the isolated nucleic acid molecules described above also are provided as well as functional equivalents, variants, analogs and fragments thereof. In one embodiment, the polypeptide is a human procollagen II N-propeptidase protein or a functionally active fragment or variants thereof.

Another embodiment of the present invention relates to, isolated, substantially purified, mammalian proteins belonging to the ADAMTS-3 subfamily. As used herein, the term “substantially purified” refers to a protein that is removed from its natural environment, isolated or separated, and at least 60% free, preferably 75% free, and most preferably 90% free from other components with which it is naturally associated.

Another embodiment of the present invention is comprised of a method of preventing, treating or ameliorating a tissue-related disorder or condition in a patient comprising the steps of administering an effective amount of ADAMTS3 or ADAMTS-3 to a site in need of repair, regeneration, or procollagen processing. This embodiment may preferably include the steps of combining an effective amount of ADAMTS3 or ADAMTS-3 and a delivery system to form a mixture; molding said mixture to form an implant; and implanting said implant into said patient. The implant is preferably suitable as a tissue substitute, and even more preferably is biodegradable. The delivery system may be comprised of any suitable delivery system including natural polymers, systhetic polymers, collagen, hydroxyapatite, calcium phosphate ceramics, bioglass, hydrogels and mixtures thereof. The effective amount of ADAMTS-2 may be delivered as a protein, a nucleic acid (e.g. a nucleic acid is selected from the group consisting of a gene, a cDNA, a vector, an RNA molecule, an antisense molecule, a ribozyme, and a peptide nucleic acid (PNA) molecule).

Embodiments of the present invention also contemplate gene therapy, wherein defective cells of a donor are genetically engineered to include an isolated nucleic acid expressing a functional ADAMTS-3 protein. The cells are then returned to the donor.

One aspect of the present invention involves the discovery and isolation of the ADAMTS3 cDNA and the corresponding ADAMTS-3 protein The expression and biological activity of the proteins are believed to be necessary for normal procollagen processing, and alteration of the expression or biological activity of these proteins may be used to influence propeptidase activity and thereby affect collagen properties. In addition, normal procollagen biosynthesis can be established by supplying a nucleic acid expressing ADMATS-3.

The invention in another aspect involves a method for decreasing procollagen propeptidase activity in a subject. An agent that selectively binds to an isolated nucleic acid molecule described herein or an expression product thereof is administered to a subject in need of such treatment, in an amount effective to decrease procollagen propeptidase activity in the subject. Preferred agents are modified antisense nucleic acids and polypeptides.

Nucleotide sequence submission. The partial sequence of ADAMTS3 (the KIAA0366 gene) was previously reported by the Kazusa DNA Institute with GenBank Accession Mo. AB002364. The novel sequence described here has been submitted to GenBank and is available with Accession No. AF247668.

### BRIEF DESCRIPTION OF THE DRAWINGS

FIG. 1A illustrates the domain organization of ADAMTS-3 and ADAMTS-2 with a key for the domains shown at the bottom of the figure;

FIG. 1B illustrates alignment of the primary structures of ADAMTS-3 and ADAMTS-2 using the single-letter amino acid code;

FIG. 2A illustrates quantitative RT-PCR assay of MRNA levels of ADAMTS-3 and ADAMTS-2 in skin fibroblasts;

FIG. 2B illustrates quantitative RT-PCR assay of mRNA levels of ADAMTS-3 and ADAMTS-2 in human cartilage;

FIG. 3 illustrates that ADAMTS-3 excites the N-propeptide of Collagen II;

FIG. 4 shows an amino acid sequence (SEQ ID NO: 1) of a full-length sequence for human ADAMTS-3;

FIG. 5 shows a nucleotide sequence (SEQ ID NO: 2) of a full-length human ADAMTS3 (coding region is in upper case) which encodes ADAMTS-3;

FIG. 6 shows an amino acid sequence (SEQ ID NO: 3) corresponding to the portion of human ADAMTS-3 corresponding to Accession No. AF247668;

FIG. 7 shows a Nucleotide sequence (SEQ ID NO. 4) corresponding to the portion of ADAMTS3 which is necessary to encode ADAMTS-3.

### DETAILED DESCRIPTION OF EMBODIMENTS OF THE INVENTION

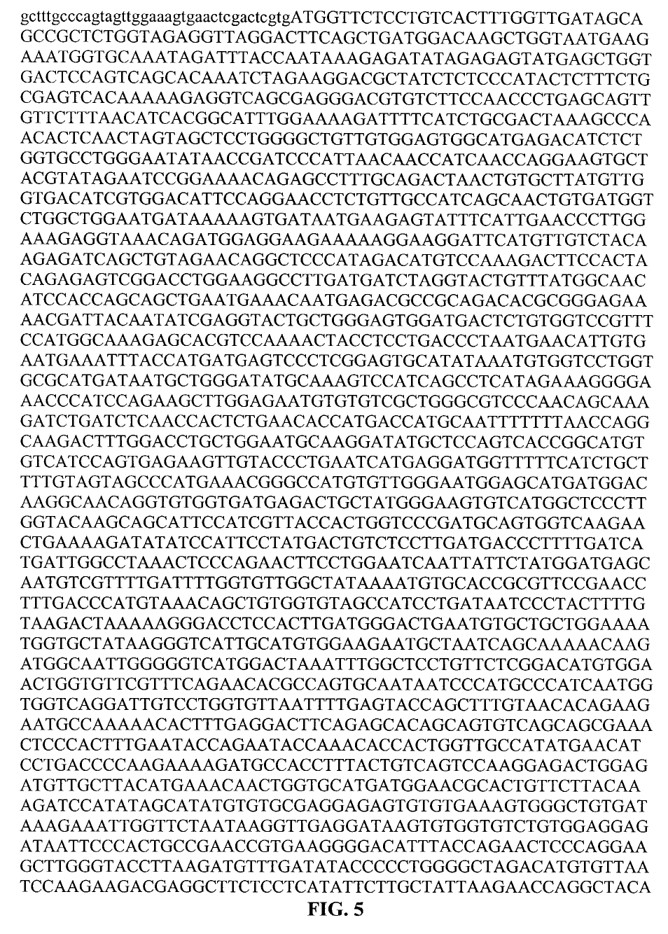

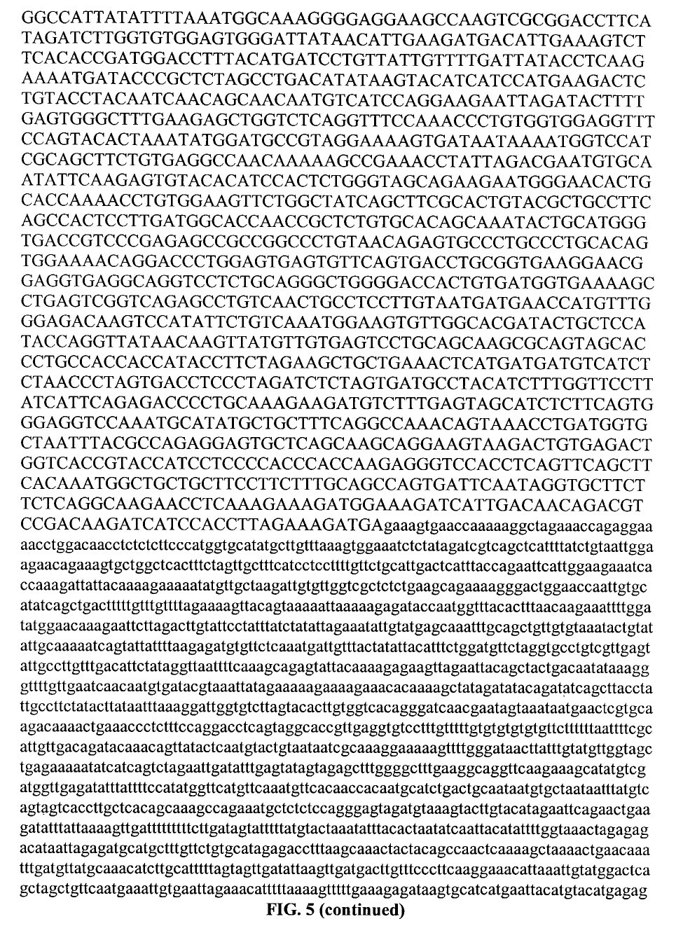

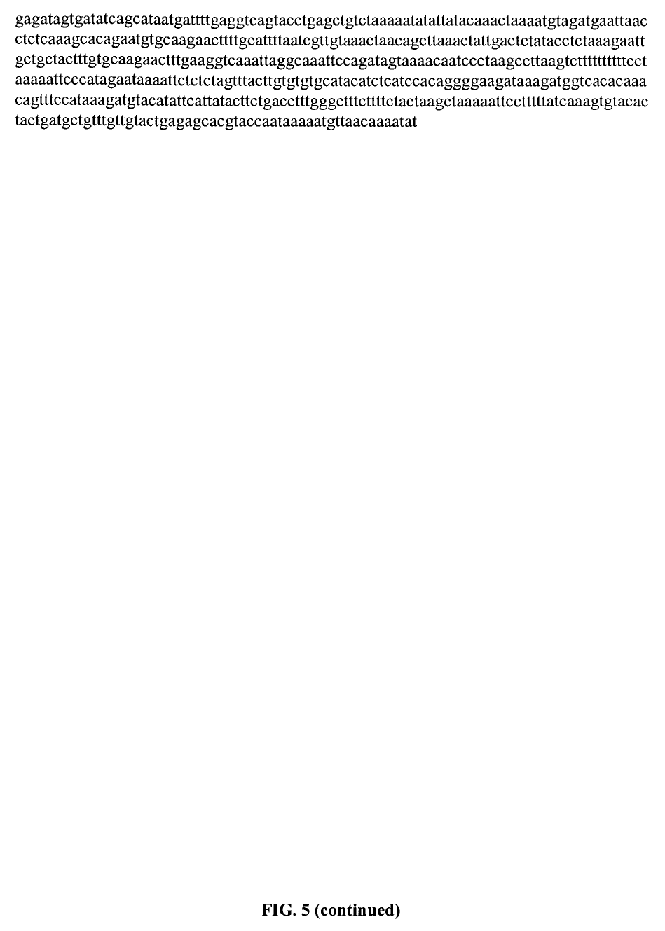

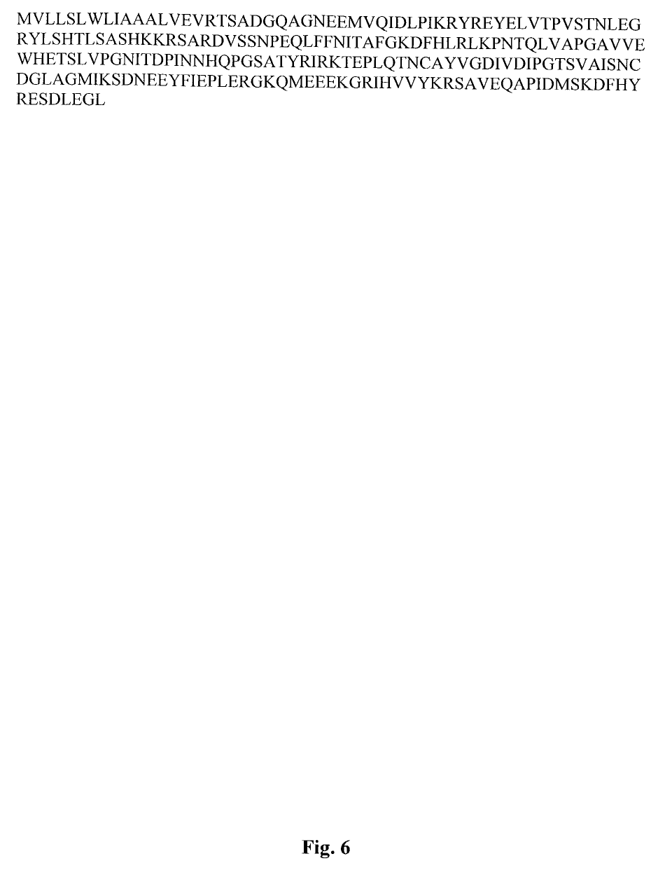

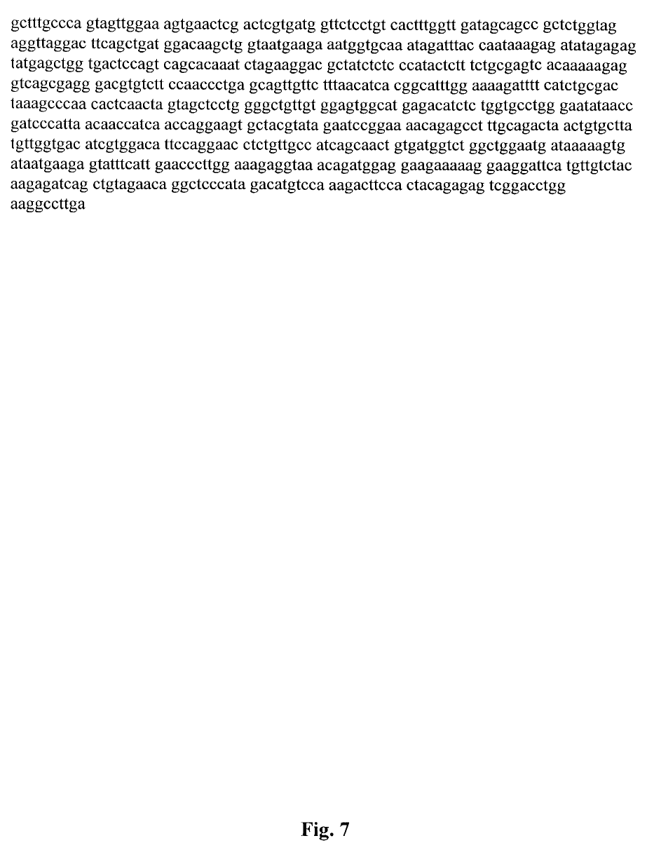

Among the various members of the ADAMTS family (currently numbering 20 gene products), the overall domain organization and amino acid sequence of ADAMTS-2 is most analogous to ADAMTS-3. ADAMTS3 cDNA was originally partially cloned from human brain and named the KIAA0366 gene. The KIAA0366 cDNA was incomplete at the 5′ end, and the translation start codon had not been identified. Through molecular cloning described herein, the complete primary structure of ADAMTS3 is identified herein as shown in SEQ ID NO: 2 and FIG. 5.

In light of the data presented here, showing that procollagen II in dermatosparactic cartilage is completely processed, it apears ADAMTS-3 (complete sequence shown in SEQ ID NO: 1 and FIG. 4) is involved in procollagen II processing. The model system used was a Swarm rat chondrosarcoma derived cell line, RCS-LTC. In monolayer culture, these cells deposit an ECM containing collagens II, IX, and XI. However, the collagen is organized into thin filaments instead of fibrils. RCS-LTC cells fail to process procollagen II beyond the stage of pN-collagen II, although the amino acid sequence of the N-propeptidase cleavage site in RCS-LTC procollagen II is normal. RCS-LTC pN-collagen II is, however, processed in vitro by addition of conditioned medium from cultures of chick chondrocytes. This suggested that RCS-LTC chondrocytes either fail to express procollagen II N-propeptidase or lack a soluble, essential cofactor. RCS-LTC chondrocytes thus provide a model system for identification of genes involved in procollagen II amino propeptide processing.

A discussed in greater detail below, transfection of RCS-LTC cells with ADAMTS3 or ADAMTS2 results in conversion of a portion of the pN-collagen II to a fully processed form. The results establish that N-propeptidase deficiency is responsible, at least in part, for defective collagen processing in RCS-LTC cells. It also appears that steady-state mRNA levels of ADAMTS2 and ADAMTS3 are different in normal human skin, in skin fibroblasts, and in cartilage, with ADAMTS-3 being expressed at higher levels than ADAMTS-2 in cartilage. Together, these data suggest that ADAMTS-3 may be a major physiological procollagen II N-propeptidase.

Thus, embodiments of the present invention may include ADAMTS subfamily protein members, particularly those which are procollagen propeptidases, genes encoding those proteins, functional modifications and variants of the foregoing, useful fragments of the foregoing, as well as therapeutics and diagnostics relating thereto. More particularly, the present invention relates to ADAMTS-3 and ADAMTS3

Homologs and alleles of the procollagen N-propeptidase genes (e.g. ADAMTS3) of the invention can be identified by conventional techniques. Thus, an aspect of the invention is those nucleic acid sequences which code for procollagen N-propeptidase proteins and which hybridize to a nucleic acid molecule consisting of ADAMTS 3, under stringent conditions, preferably highly stringent conditions. The term “stringent conditions” as used herein refers to parameters with which the art is familiar, and reference is again made to U.S. Pat. No. 6,391,610, ehich again, is incorporated herein in its entiret by refence thereto, particularly with regard to the definitions of “stringent” and “highliy stringent”. The skilled artisan will be familiar with such conditions, and thus they are not given here. It will be understood, however, that the skilled artisan will be able to manipulate the conditions in a manner to permit the clear identification of homologs and alleles of procollagen N-propeptidase proteins of the invention. The skilled artisan also is familiar with the methodology for screening cells and libraries for expression of such molecules which then are routinely isolated, followed by isolation of the pertinent nucleic acid molecule and sequencing.

The invention also provides isolated unique fragments of ADAMTS3. A unique fragment is one that is a ‘signature’ for the larger nucleic acid. Unique fragments can be used as probes in Southern blot assays to identify family members or can be used in amplification assays such as those employing PCR. Unique fragments also can be used to produce fusion proteins for generating antibodies or for generating immunoassay components. Likewise, unique fragments can be employed to produce fragments of the procollagen N-propeptidase protein, useful, for example, in immunoassays or as a competitive inhibitor of the substrate of the procollagen N-propeptidase protein in therapeutic applications. Unique fragments further can be used as antisense molecules to inhibit the expression of the procollagen N-propeptidase proteins of the invention, particularly for therapeutic purposes as described in greater detail below.

As mentioned above, the invention embraces antisense oligonucleotides that selectively bind to a nucleic acid molecule encoding an procollagen N-propeptidase protein, to decrease procollagen N-propeptidase activity (particularly procollagen I N-propeptidase activity). This is desirable in virtually any medical condition wherein a reduction in collagen production activity is desirable.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene.

Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The invention also involves expression vectors coding for procollagen N-propeptidase proteins (particularly procollagen II N propeptidase proteins) and fragments and variants thereof and host cells containing those expression vectors. Virtually any cells, prokaryotic or eukaryotic, which can be transformed with heterologous DNA or RNA and which can be grown or maintained in culture, may be used in the practice of the invention. Examples include bacterial cells such as E.coli and mammalian cells such as mouse, hamster, pig, goat, primate, etc. They may be of a wide variety of tissue types.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g. β-galactosidase or alkaline phosphatase), and genes which visibly affect the phenotype of transformed or transfected cells, hots, colonies or plaques. Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not: result in the introduction of a frame-shift mutation; interfere with the ability of the promoter region to direct the transcription of the coding sequences; or interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

Expression vectors containing all the necessary elements for expression are commercially available and known to those skilled in the art. See Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding the procollagen N-propeptidase protein or fragment or variant thereof. That heterologous DNA (RNA) is placed under operable control of transcriptional elements to permit the expression of the heterologous DNA in the host cell. In still another aspect of the invention, a defective chondrocyte or fibroblast or precursor thereof is treated with DNA in a manner to promote via homologous recombination intracellularly the correction of a defective procollagen N-propeptidase gene.

The invention also contemplates screening assays to detect the presence or absence of the procollagen N-propeptidase protein and in purification protocols to isolate procollagen N-propeptidase proteins.

When used therapeutically, the compounds of the invention are administered in therapeutically effective amounts. In general, a therapeutically effective amount means that amount necessary to delay the onset of, inhibit the progression of, or halt altogether the particular condition being treated. Therapeutically effective amounts specifically will be those which desirably influence procollagen N-propeptidase activity, be it inhibiting or enhancing procollagen N-propeptidase I or enhancing procollagen N propeptidase II. When it is desired to decrease procollagen N-propeptidase I/II activity, then any inhibition of procollagen N-propeptidase activity is regarded as a therapeutically effective amount. When it is desired to increase procollagen N-propeptidase I/II activity, then any enhancement of procollagen N-propeptidase activity is regarded as a therapeutically effective amount. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences; 18th edition, 1990, pp 1694-1712; incorporated by reference). Those of skill in the art can readily determine the various parameters and conditions for producing antibody aerosols without resort to undue experimentation. When using antisense preparations of the invention, slow intravenous administration is preferred.

The invention also contemplates gene therapy. The procedure for performing ex vivo gene therapy is well known in the art. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art In vivo gene therapy using vectors such as adenovirus also is contemplated according to the invention.

In vivo gene therapy can also be used for systemic treatement, an area in which gene therapy has broad applications. Systemic treatment involves transfecting target cells with the DNA of interest, expressing the coded protein in that cell, and the capability of the transformed cell to subsequently secrete the manufactured protein into blood.

A variety of methods have been developed to accomplish in vivo transformation including mechanical means (e.g. direct injection of nucleic acid into target cells or particle bombardment), recombinant viruses, lipsomes, and receptor-mediated endocytosis (RME) (for reviews, see Chang et al. 1994 Gastroenterol. 106:1076-84; Morsy et al. 1993 JAMA 270:2338-45; and Ledley 1992 J. Pediatr. Gastroenterol. Nutr. 14:328-37).

The following examples, materials, methods, experimental procedures, discussion, and detailed description are meant to further illustrates the present invention but, of course, should not be construed as in any way limiting its scope.

Materials and Methods, & Experimental Procedures: Cloning of ADAMTS3. The previously reported 5774 bp KIAA0366/ADAMTS3 cDNA (23) was incomplete at its 5′. The sequence was extended further in the 5′ direction by RACE using the Marathon™ system, and Marathon™ human fetal brain cDNA (reagents from Clontech, Palo Alto) as template, essentially as previously described. PCR was done with nested ADAMTS3 specific antisense oligonucleotide primers 5′TCAAGGCCTTCCAGGTCCGACTCTC3′ and 5′GGGAGCCTGTTCTACAGCTGATCTC3′ and with nested adaptor primers at the 5′ end of the template. The RACE products were cloned and sequenced as previously described.

Generation of ADAMTS3 and ADAMTS2 expression constructs. To generate a cDNA construct for expression of full length ADAMTS-3, we first deleted the 5′ end of the KIAA0366 cDNA (in pBluescript II SK+ [Stratagene, La Jolla], provided by Dr. Takahiro Nagase of the Kazusa DNA Institute). The deleted segment extended from the 5′ Sal I cloning (i.e., vector) site up to a unique internal AccI site at nt position 598 (KIAA0366 sequence enumeration). We replaced this fragment with a PCR-derived fragment of ADAMTS3 cDNA extending from the 5′ untranslated sequence to just downstream of the AccI site. Briefly, PCR was performed with Advantage PCR reagents (Clontech, Palo Alto, Calif.), using the RACE cDNA clone as template, the forward primer 5′AACTCGAGGAAAGTGAACTCGACTCGTG3′ (XhoI site underlined) and reverse primer 5′AGCCTGTTCTACAGCTGATC3′. The resulting amplicon was digested with XhoI and AccI (at the internal AccI site) and cloned into the SalI-AccI restricted KIAA0366 cDNA. This introduced the authentic ADAMTS3 ribosome binding sequence, translation start codon and complete signal peptide into the KIAA0366 cDNA. This insert encoding full-length ADAMTS-3 was excised from pBluescript with XhoI and NotI and ligated into the corresponding sites in pcDNA 3.1 (+) myc-His A (Invitrogen, San Diego, Calif.). In this mammalian expression construct, ADAMTS-3 is not in frame with the C-terminal myc and poly-histidine tags. For expression of ADAMTS-2, three overlapping bovine cDNA clones which have been previously reported were appropriately digested (with NotI, BclI, EagI, and KpnI) and assembled to generate a construct encoding full-length bovine ADAMTS-2. The ADAMTS2 cDNA was inserted into the NotI/XbaI sites of the pcDNA3 expression vector (Invitrogen, San Diego, Calif.) using a XbaI adapter. These were kindly provided by Dr. A. C. Colige.

Isolation of RNA. Skin samples obtained from the forearm of healthy volunteers were used to isolate dermal fibroblasts or were stored in liquid nitrogen until use (Laboratoire de Biologie des Tissus Conjonctifs, Sart-Tilman, Belgium, Ethics Committee Approval F94/14/1871). Dermal fibroblasts grown from skin explants were cultivated in Dulbecco's Minimum Essential Medium (DMEM) supplemented with 10% fetal bovine serum (FBS). For northern analysis, total RNA was purified from skin samples pulverized in liquid nitrogen or from dermal fibroblasts in culture, after solubilization of homogenates and cells in 0.1M Tris HCl, pH 7.5, 5M guanidium thiocyanate, 1% β-mercaptoethanol. These extracts were centrifuged at 100,000×g for 18 h on a cesium chloride (CsCl 2 ) cushion (0.01 M ethylene diamine tetra-acetic acid (EDTA), pH7.5, 5.7 M CsCl 2 ). MRNA was subsequently purified using PolyATract mRNA Isolation System (Promega Benelux B.V.), according to manufacturer's instructions. For quantitative RT-PCR, RNA was harvested from human fetal cartilage or from dermal fibroblasts using Trizol (Life Technologies, Rockville, Md.) and manufacturer's recommended protocols. DNA was eliminated by treatment with DNase I (DNA-FreeTM, Ambion, Austin, Tex.).

Quantitative reverse transcriptase-PCR (RT-PCR) analysis. Total RNA (2.5 mg) was reverse-transcribed with the SuperScript First-Strand Synthesis System for RT-PCR (Life Technologies, Rockville, Md.) using oligo-dT as a primer. Real-time PCR was performed in an ABI Prism 7700 Sequence Detector using SYBR Green PCR Core Reagents (Applied Biosystems, Foster City, Calif.). In this system, continuous, automated quantitation of the PCR product is performed by measuring the fluorescence generated by the binding of SYBR Green to double-stranded DNA. All PCR amplifications were performed in triplicate along with parallel measurements of GAPDH cDNA (an internal control). Data were analyzed according to the comparative C t method (Applied Biosystems protocols) and represented after normalization to GAPDH levels. To confirm these data, PCR products were also separated on a 2.0 % agarose gel, visualized with UV light through a SYBR Green filter and photographed. The following primers were used for amplification at a concentration of 300 nM: ADAMTS2,

5′TGGGAAGCACAACGACATTG3′(forward) and

5′CTCGGTCGTCGAGGGATTAG3′(reverse), ADAMTS3,

5′TCAGTGGGAGGTCCAAATGCA3′(forward) and

5′GCAAAGAAGGAAGCAGCAGCC3′(reverse), GAPDH,

5′CCACTGCCAACGTGTCAGTGG3′(forward) and

5′AAGGTGGAGGAGTGGGTGTCG3′(reverse). As an additional control, RT-PCR was also performed in the absence of template.

Northern analysis of ADAMTS2 and ADAMTS3 expression. A commercially available adult human multiple tissue northern blot and a mouse embryo northern blot (Clontech Inc. Palo Alto, Calif.) were hybridized as per manufacturer's instructions using ExpressHyb™ hybridization fluid (Clontech, Palo Alto, Calif.). The following cDNA probes were used after random-primed labeling with [α³²P]-dCTP: a fragment containing nucleotides 946-1379 of human ADAMTS-2 cloned in pCR4-TOPO (for human multiple tissue northern blot); a 1.1 kbp HindIII fragment from the KIAA0366/ADAMTS3 cDNA (for human multiple tissue northern blot); the insert of IMAGE clone 1246561, available with GenBank accession no. AA832579 (mouse ADAMTS-2 probe for mouse embryo northern blot); the insert of IMAGE clone 727026, available with Accession no. AA402760 (mouse ADAMTS-3 probe for mouse embryo northern blot). Exposure of the blots to X-ray film was for 3-7 days.

Poly-A+ RNA (0.8 μg) from human skin or human skin fibroblasts was electrophoresed on a formaldehyde-agarose gel and blotted to Hybond N⁺ nylon membrane (Amersham-Pharmacia Biotech). cRNA probes for human ADAMTS2 and ADAMTS3 were generated by transcription from the respective clones using the Strip-EZ RNA kit (Ambion), T3 RNA polymerase and [α³²P]-UTP as per manufacturer's instructions. Prehybridization (1 hour) and hybridization (18 hours) were performed at 65° C. in 0.2 M Na₂HPO₄ (pH 7.2), 1 mM EDTA, 1% bovine serum albumin, 7% sodium dodecyl sulfate (SDS) and 20% formamide. Stringency washes were carried out at 65° C. in 40 mM Na₂HPO₄, 1 mM EDTA and 1% SDS.

Cell Culture and stable transfection of RCS-LTC chondrocytes. Monolayer cultures of RCS-LTC cells were maintained in DMEM containing 4.5 g/liter glucose (Life Technologies, Grand Island, N.Y.) and 10% FBS (Hyclone Labs, Logan, Utah) at 37° C. in 5% CO₂. Culture medium was changed every other day and confluent cultures were sub-cultured every 2 weeks as described previously. Lipofectamine Plus (Life Technologies, Grand Island, N.Y.) was used for RCS-LTC transfections. Cells were plated at a density of 3×10⁵ cells/well in 6 well plates (Falcon Franklin Lakes, N.J.). After 24 h, the wells were rinsed with OPTI-MEM (Life Technologies, Grand Island, N.Y.) and transfected with the human ADAMTS3 or bovine ADAMTS2 cDNA constructs mg DNA/well) as per the manufacturer's instructions. As a control for efficiency of transfection and to provide a negative control for procollagen II processing, cells were separately transfected with pcDNA3.1/Myc-His (+)lacZ encoding the E. coli lacZ gene. Mock transfections were performed without cDNA as a control for efficacy of antibiotic selection. After 4 days in culture, transfected cells were selected in media supplemented with 1 mg/ml G418 sulfate (Geneticin, Life Technologies, Grand Island, N.Y.). After 2 weeks in culture, the chondrocytes from the mock transfections did not survive selection. Geneticin-resistant ADAMTS3, ADAMTS2, and lacZ transfected chondrocytes were expanded and maintained as pools in serial monolayer culture as previously described for the RCS-LTC cell line, but in the continued presence of 1 mg/ml Geneticin.

β-Galactosidase expression in the lacZ stable transfectants was detected histochemically by staining the cells with 5-bromo-4-chloro-3-indolyl-β-D-galactoside (X-gal, Life Technologies, Grand Island, N.Y.). Briefly, the cultures were rinsed with phosphate-buffered saline (150 mM NaCl, 15 mM sodium phosphate, pH 7.3), fixed with 0.2% glutaraldehyde and incubated with 1 mg/ml X-gal, 5 mM potassium ferricyanide, 5 mM potassium ferrocyanide and 2 mM MgCl₂ in PBS for 24 h at 37° C.

Procollagen analysis in RCS-LTC cells. ADAMTS3, ADAMTS2 and lacZ stable transfectants as well as untransfected RCS-LTC chondrocytes were grown in 6 well plates (Falcon, Franklin Lakes, N.J.) following sub-culture as described. For the last day of culture, the culture medium was supplemented with 10 mg/ml L-ascorbate and 100 mg/ml β-aminoproprionitrile. Cells and ECM were rinsed with PBS and extracted with boiling Laemmli sample buffer containing 100 mM dithiothreitol (DTT) or with 0.4 M NaCl, 50 mM Tris HCl, pH 7.5 containing 5 mM EDTA, 1 mM PMSF, 0.1 mM benzamidine, and 0.1% Triton X-100. To identify procollagen II processing intermediates, untransfected RCS-LTC chondrocytes were grown for 4 days in the absence of ascorbate and β-aminopropionitrile. The cell layers were extracted as above. Extracts were repeatedly passed through a 27.5 G needle to reduce viscosity and were centrifuged at 15,000 rpm for 30 minutes at 4° C.

Collagen II in extracts of cartilage and RCS-LTC transfectants was analyzed by gel electrophoresis and immunoblotting. Extracts were heated to 100° C. for 3 minutes in Laemmli sample buffer containing 100 mM DTT. Collagen II chains were resolved by 6% polyacrylamide gel electrophoresis and detected after transfer to polyvinyl difluoride (PVDF) membrane (BioRad, Hercules, Calif.) using a monoclonal antibody to collagen II (1C10) at a dilution of 1:2000 and the Renaissance western blot detection reagent (NEN Lifescience Products, Boston, Mass.). Monoclonal antibody 1C10 (D. R. Eyre, unpublished data) recognizes an epitope in denatured α1(II) CB9, 7. It detects procollagen II as well as processed collagen II on western blots.

As controls, fully processed collagen II from a 4M guanidine HCl extract of human fetal cartilage and pepsinized collagen II from the RCS-LTC cell line were used.

Procollagen cleavage analysis in dermatosparactic cartilage. Nasal cartilage from a dennatosparactic cow and normal fetal bovine epiphyseal cartilage were extracted in 4 M guanidine HCl, 50 mM Tris-HCl, pH 7.4 with 2 mM EDTA, 5 mM benzamidine, 2 mM PMSF and 10 mM 1,10 phenanthroline at 40° C. for 24 h. Collagen in this extract was separated by reducing SDS-PAGE gel electrophoresis and visualized by Coomassie blue staining and by western blot analysis using 1C10 as described above.

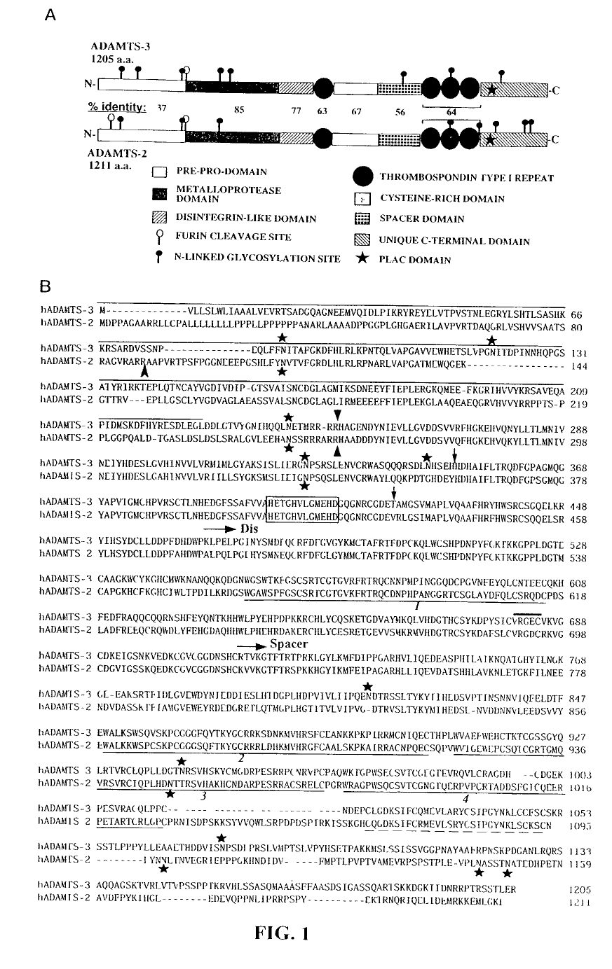

Molecular cloning of full length ADAMTS-3 and comparison with ADAMTS-2. Using 5′ RACE, a novel 720 bp cDNA clone encoding the 5′ untranslated region,the translation start codon, signal peptide, and pro-domain of ADAMTS-3 was obtained. The novel 5′ sequence we obtained has not been previously described and is deposited in GenBank with Accession No. AF247668. When incorporated into the KIAA0366 sequence, we were therefore able to identify the complete open reading frame (ORF) and conceptual translation product of ADAMTS3. The predicted start codon is the 5′ most methionine codon (ATG) in the ORF (FIG. 1B) and is preceded by an in-frame stop codon, 16 nucleotides upstream. It is located within the context of a suitable Kozak consensus sequence for translation (contains purine [G] at position −3 with respect to the A of the ATG codon, and G at position +4). The predicted ADAMTS-3 protein (1205 amino acids) is comparable in length to human and bovine ADAMTS-2 (1211 and 1205 amino acids respectively). The predicted mature (furin-processed) forms of these proteases are also of comparable length, 957 residues (ADAMTS-3) and 953 residues (bovine and human ADAMTS-2) long. ADAMTS3 predicts a full-length protein of molecular mass 135.6 kDa and a furin processed form of 107.5 kDa. A number of N-linked glycosylation sites are predicted by the sequence (see below and FIG. 1) and thus post-translational modification is likely to increase the molecular mass of ADAMTS-3.

FIG. 1A shows the amino acid identity (in percent) between ADAMTS-3 and ADAMTS-2 for each domain (except the C-terminal domain). As can be seen in FIG. 1A, ADAMTS-3 and ADAMTS-2 have a similar domain structure. As can also be seen in FIG. 1B, ADAMTS-3 and ADAMTS-2 have and an overall sequence identity of 61%. From amino to carboxyl terminus, each of these enzymes consists of the following domains (with percent sequence identity in parentheses): signal peptide, pro-domain (37%) demarcated from the catalytic domain (85%) by a furin cleavage site, disintegrin-like domain (77%), central thrombospondin type I (TS) repeat (63%), cysteine-rich domain (67%), spacer domain (56%), three additional TS repeats (64%), followed by a unique, mostly nonhomologous C-terminal extension. The likely furin processing site for generation of mature ADAMTS-3 and ADAMTS-2 is indicated by the arrowhead in FIG. 1B. In FIG. 1B, the sequence encoded by the RACE clone is overlined; TS repeats are underlined and indicated by numbers; the zinc-binding histidine triad is enclosed in a box; and the boundaries of a region of complete amino acid sequence identity in the catalytic domain are indicated by the vertical arrows. The N-termini of the disintegrin-like (Dis) and spacer domains are indicated. The cysteine-rich domain extends from TS repeat 1 to the start of the spacer domain. Potential cell-binding RGD/E sequences are indicated by the thick overline. The PLAC domains are shown by the dashed underline. Potential sites for N-linked glycosylation are indicated by asterisks.

The presumed furin cleavage sites in ADAMTS-3 and ADAMTS-2 are at identical locations. ADAMTS-3 and ADAMTS-2 catalytic domains are strikingly similar, demonstrating complete sequence identity (but for one amino acid) in a region of 69 amino acids which includes the zinc-binding active site sequence. ADAMTS-3 and ADAMTS-2 each have eight consensus sites for potential N-linked glycosylation. Of these, four are conserved absolutely between ADAMTS-2 and ADAMTS-3, two conserved sites being in the pro-domain, and one site each in the catalytic domain and the second TS domain. In contrast to ADAMTS-2, which contains the potential cell binding sequence CVRGDC, ADAMTS-3 has the sequence CVRGEC at the corresponding location. The C-terminal domains of these two molecules are of comparable length (184 and 191 amino acids in ADAMTS-2 and ADAMTS-3, respectively), but show little sequence similarity other than a highly conserved PLAC (Protease and LACunin) domain (83% identity). The PLAC domain was first described in an insect protein, lacunin, which contains all the ancillary domains of ADAMTS, and can therefore be considered an ADAMTS-like protein. The PLAC domains of human ADAMTS-3 and bovine ADAMTS-2 each contain six cysteine residues. In a previously published human ADAMTS-2 sequence (GenBank Accession No. AJ003125), one of these cysteines (at position 1090 in FIG. 1B) was substituted by serine. However, it is likely that this represents a sequence variation or error since there is a cysteine at this position in three independent ADAMTS2 EST sequences (GenBank accession nos. AI417257, AI624388 and AI089232).

ADAMTS-2 and ADAMTS-3 constitute a structurally distinctive procollagen N-propeptidase (PCNP) subfamily of the ADAMTS family. ADAMTS-2 and ADAMTS-3, as a sub-group, are distinct from the rest of the ADAMTS family in a number of ways. In terms of domain organization, these are the only two members of the ADAMTS family to have three C-terminal TS domains. Furthermore, they are the only two members of the ADAMTS family to have a substantial C-terminal extension downstream of these TS domains. The location of the PLAC domain within this C-terminal extension is also unique, because in other ADAMTS family members where it is present, such as in ADAMTS-7B and ADAMTS-10 it is usually at the very carboxyl end of the protein. In the PCNP sub-family, the PLAC domain is internal.

A number of sequence hallmarks are unique to the PCNP subfamily of enzymes, including: the pro-domain contains only two cysteines, in contrast to other ADAMTS enzymes, which usually contain three; and the catalytic domain contains 6 cysteines as opposed to eight for the other ADAMTS. In other members of the ADAMTS family, the usual arrangement consists of five cysteines upstream of the zinc-binding site and three downstream. In the PCNP subfamily, there are only three cysteines upstream of the zinc-binding sequence. The three downstream cysteines are however, at absolutely conserved positions with regard to other ADAMTS enymes. This arrangement of cysteines suggests that the catalytic domain of the PCNP subfamily may be structurally different from that of the other ADAMTS enzymes. Additionally, the sequence of the zinc-binding triad (HETGHLGMEHD) in the PCNP subfamily is unique in that it contains threonine in the third position (underlined), whereas all other ADAMTS enzymes have a hydrophobic residue with a long side-chain (leucine or isoleucine) at this position; and the spacer domains of the ADAMTS family are quite variable in length and sequence. However, those of the PCNP subfamily are significantly similar to each other (56% amino-acid identity).

On the basis of domain and amino acid sequence homology, this subfamily appears to contain no more than three members as determined by a search of the completed human genome sequence (Celera Genomics, Rockville, Md.). We designate the “classical” PCNP, ADAMTS-2, as PCNP1, and ADAMTS-3 as PCNP2, in keeping with the practice of providing trivial nomenclature which reflects the substrate of the enzyme.

Differential tissue-specific expression of ADAMTS2 and ADAMTS3. Adamts2 and Adamts3 were both expressed during mouse embryogenesis. Adamts2 expression was noted in mouse embryos at 7, 15 and 17 days. Two mRNA species (7.8 kb and 4.0 kb) were detected. A single Adamts3 mRNA species (˜7.2 kb in size) was also detected in mouse embryos at 7, 15 and 17 days of gestation. Restricted expression of ADAMTS2 and ADAMTS3 was seen in the eight normal human adult tissues examined by northern analysis. In human placenta, lung and liver, two ADAMTS2 transcripts were present, migrating at approximately 7.8 kb and 4.0 kb as previously described. The previously described 2 kb transcript which encodes a truncated form of ADAMTS-2 was not detected. Highest expression of ADAMTS3 was noted in placenta with lower level expression in lung, brain and heart, with a single mRNA species migrating at approximately 7.0 kb.

In skin samples and in skin fibroblasts, northern analysis with equivalent amounts of cRNA probes for ADAMTS2 or ADAMTS3 demonstrated a differential prevalence of steady-state mRNA levels. Autoradiograms generated by 1 h (ADAMTS2) and 18 h exposure (ADAMTS-3) illustrated this point. Based on these different durations of exposure, it is concluded that a substantially stronger signal is obtained with an ADAMTS2 probe than with ADAMTS3 in skin and skin fibroblasts, suggesting that they contain higher steady-state levels of ADAMTS2 mRNA than ADAMTS3 mRNA. All three previously reported ADAMTS2 transcripts (7.0 kb, 4.0 kb and 2.0 kb) as well as some other minor transcripts were seen in skin fibroblasts, while only the 4.0 kb and 2.0 kb mRNAs were found in skin. However, with the ADAMTS3 probe, a 4.8 kb band was identified, plus 2.3 kb band in skin fibroblasts. The discrepancy of these ADAMTS-3 bands with those noted in multiple tissue northerns is presently unexplained, but it is possible, that like ADAMTS2, ADAMTS3 also generates multiple transcripts in a tissue specific fashion. It was also noted that while ADAMTS3 signal is stronger in skin as opposed to skin fibroblasts, the reverse was true for ADAMTS2.

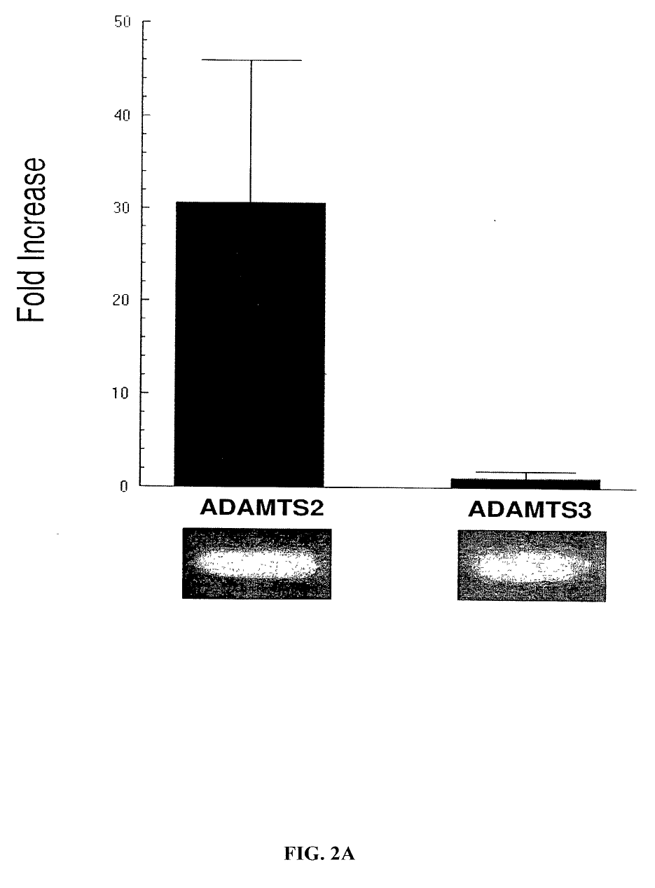

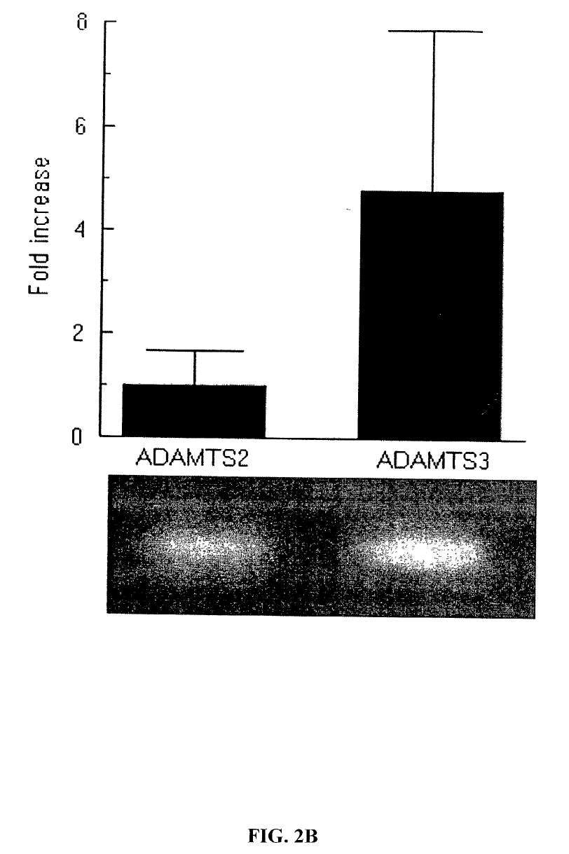

To obtain a quantitative estimate of relative mRNA levels, quantitative RT-PCR analysis of ADAMTS2 and ADAMTS3 mRNA in cultured skin fibroblasts and human cartilage was performed. The data demonstrate considerably higher levels of steady-state ADAMTS2 mRNA levels in human skin fibroblasts relative to ADAMTS3. In contrast, as shown in FIG. 2A and FIG. 2B quantitative RT-PCR analysis of RNA from human fetal cartilage demonstrates an approximately five-fold higher steady-state level of ADAMTS-3 mRNA compared to ADAMTS2. In FIGS. 2A and 2B, the mean and standard deviation of three different PCR reactions is shown, as well as gel electrophoresis of representative PCR reactions (inset).

Procollagen II is completely processed in dermatosparactic cartilage. Dermatosparactic animals have no functioning ADAMTS-2. Despite this, Coomassie-blue staining of collagen extracted from dermatosparactic nasal cartilage demonstrated the presence of collagen chains which migrated similarly to the processed collagen II chains in control cartilage. This was confirmed by western blot analysis of these extracts using a monoclonal antibody which recognizes procollagen II as well as processed collagen II. This analysis showed that essentially all of the immunoreactive collagen was fully processed. Some pN-collagen I was visible in the extracts from dermatosparactic cartilage, confirming the origin of this tissue.

ADAMTS3 and ADAMTS2 process procollagen II in transfected RCS-LTC cells. The efficiency of RCS-LTC transfection was monitored by β-galactosidase staining. LacZ-transfected cells also served as a negative control for analysis of procollagen processing. FIG. 5A (panels a and b) shows lacZ-transfected chondrocytes on day 2 and day 8 after subculture, following transfection, Geneticin selection and histochemical staining for β-galactosidase activity. Cells stably transfected with lacZ showed dark blue staining (representing about 10% of the population) while no staining was seen in the ADAMTS3 and ADAMTS2 stably transfected populations as expected (FIG. 5A, panels c and d). A similar efficiency of transfection and/or expression was assumed for the ADAMTS3- and ADAMTS2-transfected cells as for the lacZ-transfected cells.

To determine if ADAMTS-3 was capable of enzymatically removing the N-propeptide of procollagen II, lysates of ADAMTS3-, ADAMTS2- and lacZ-transfected RCS-LTC chondrocytes were blotted and procollagen II and collagen II were identified using monoclonal antibody 1C10. The results show some processing of pN-collagen II to mature collagen II in the ADAMTS2- and ADAMTS3-transfected cells, but none in lacZ-transfected cells or in untransfected cells. Following N-propeptide excision, the a1(II) chains migrate faster than the pN-collagen II chains and at a position similar to the naturally processed a1(III) chain or pepsinized collagen II.

Procollagen I processing in dermatosparaxis is most deficient in skin, although mature skin has some processed collagen. Many collagen I-containing dermatosparactic tissues such as tendon, ligament, sclera and aorta show the presence of significant amounts of fully processed collagen I. None of these tissues, nor bone, which relies on collagen I for its mechanical strength, have been noted to be fragile. Very recently, Adamts2 knockout mice have been reported to have significant amounts of processed collagen in skin.

These anomalies were attributed to the presence of residual pN-collagen processing activity, due to either the incompleteness of the genetic defect, or to compensation by another enzyme. The demonstration that the causative mutations were functionally null favored the existence of one or more additional procollagen N-propeptidases. The presence of processed procollagen I in many dermatosparactic tissues, including skin, suggested that this putative alternative propeptidase(s) might be regulated differently from ADAMTS-2 in skin and other tissues or that it may not be as efficient in procollagen I processing as ADAMTS-2.

ADAMTS-2 can process procollagen II. Therefore, we expected that procollagen II processing would be abnormal in dermatosparactic cartilage. However, it appears that collagen II is normally processed in dermatosparactic cartilage, despite the absence of ADAMTS-2. This leads to the conclusion that enzyme(s) other than ADAMTS-2 that could process procollagen II exist (implication ADAMTS-3). Although EDS-VIIC patients are of short stature, they do not have chondrodysplasia or premature arthritis. A defect in collagen II processing similar to the collagen I processing defect in dermatosparactic skin would be expected to cause a severe chondrodysplasia, given the critical role of collagen II in structural stability of cartilage matrix. Our studies provide an explanation for the absence of cartilage fragility and/or chondrodysplasia in dermatosparaxis or EDS-VIIC. Our data suggests the presence of an alternative pathway of procollagen II processing. The presence of procollagen I in dermatosparactic nasal cartilage, but not in normal articular cartilage may be explained by the differences in composition of these cartilages, the inclusion of perichondrium in the extract, or by upregulation of collagen I gene expression, which has been previously noted in dermatosparaxis. In contrast to our finding, the recently described Adamts2 knockout mice retain some unprocessed collagen II in their cartilage.

ADAMTS-2 and ADAMTS-3 comprise a structurally and functionally distinct subfamily of ADAMTS proteases. ADAMTS-3 and ADAMTS-2 are closely related in the length of the polypeptide chains, and their primary sequence and domain organization, but are located on different chromosomes. We previously mapped ADAMTS3 to human chromosome 4, distinct from the ADAMTS2 locus on human chromosome 5. While the close similarities in their catalytic domains suggest similar catalytic mechanisms, greater differences in their ancillary domains (i.e., the TS, disintegrin-like, cysteine rich and spacer domains) may affect substrate preferences or binding and compartmentalization in ECM. For example, ADAMTS-1 ancillary domains are responsible for ECM-binding and the TS domains of aggrecanase-1 (ADAMTS-4) are required for binding to native aggrecan. A splice variant of ADAMTS2 which generates a short form of ADAMTS-2 lacking the ancillary domains is functionally inactive in procollagen I processing.

Gene regulation can also determine which of two or more related genes are functional in any given tissue. Multiple tissue northern blots demonstrated that ADAMTS2 and ADAMTS3 are differentially regulated in various tissues. Our results indicated that the steady-state mRNA levels of ADAMTS2 were substantially higher than ADAMTS-3 in RNA isolated from skin and even more so in RNA isolated from skin fibroblasts. Data from northern blot analysis was supported by real-time, quantitative RT-PCR analysis of skin fibroblast RNA. In contrast, in human cartilage, ADAMTS-3 levels were over four-fold higher than those of ADAMTS-2.

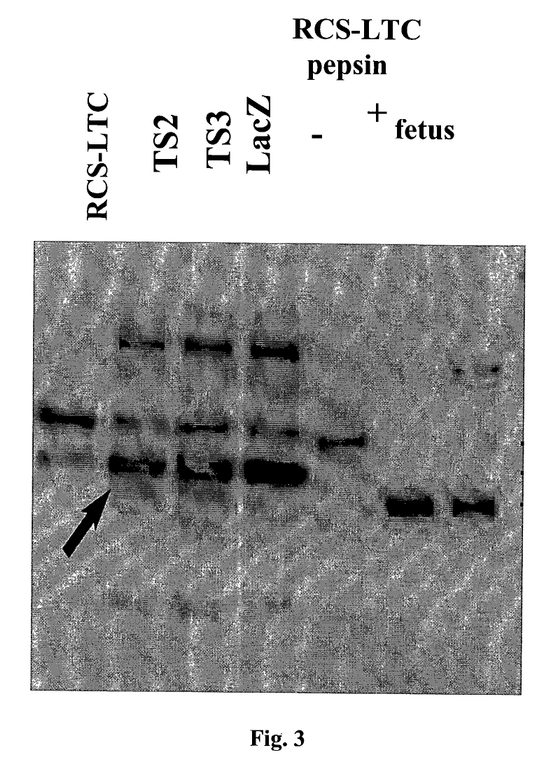

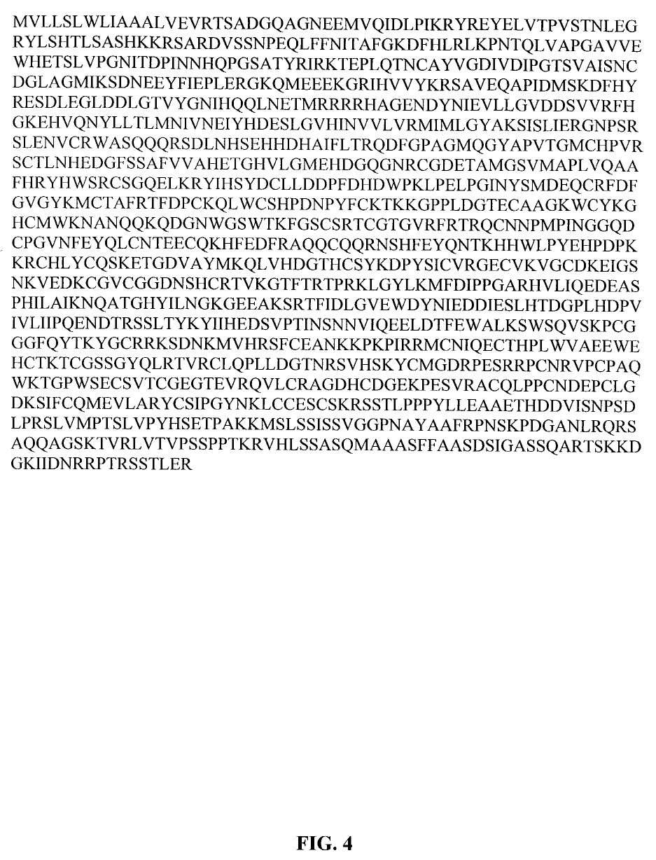

Transfection with ADAMTS2 and ADAMTS3 leads to procollagen II processing in RCS-LTC cells. We have taken a genetic approach to identify a function for ADAMTS3 and to determine the nature of the underlying processing defect in RCS-LTC cells. The system was RCS-LTC cell line with stable transfection (pools) and collagen II Mab 1C10 (Eyre). As shown in FIG. 3, transfection of these cells with ADAMTS2 and ADAMTS3, but not with lacZ (a negative control), results in processing of pN-collagen II to the fully processed form. The controls were (+) +Pepsinized RCS-LTC collagen; +Fetal collagen II; −RCS-LTC collagen II; and +ADAMTS-2 transfection. Generation of processed collagen II was evidenced by co-migration of the processed form with pepsinized RCS-LTC procollagen II (pepsin removes noncollagenous propeptides) and with native collagen II isolated from human cartilage. This suggests that ADAMTS-3, like ADAMTS-2, has pN-collagen II processing activity. Our results suggest that in quantitative terms it is roughly equivalent to that of ADAMTS-2. Given the fact that ADAMTS-2 is an established procollagen I/II processing enzyme and that there is a high degree of similarity of ADAMTS-3 to ADAMTS-2, it is very likely that ADAMTS-3 directly processes procollagen II. It may also serve as a target for inhibitors or competitors of ADAMTS-2.

There are several possible explanations for the persistence of substantial amounts of pN-collagen II in ADAMTS2- or ADAMTS3-transfected cells. The transfected cells were maintained as a pooled population rather than as clonally selected lines so that there may be transfected cells which do not express the construct at all or do so at low levels. To support this possibility, only a small proportion of lacZ-transfected cells was found to express β-galactosidase activity. While not wishing to be bound by theory, it is unlikely that cells not containing the cDNA constructs survive because control, untransfected cells subjected to Geneticin selection pressure were killed after 2 weeks.

The defect in RCS-LTC cells suggests a failure to produce a functional processing enzyme. This could result from a structural mutation in ADAMTS-2 or ADAMTS-3 or because of transcriptional repression of these genes in RCS-LTC cells. While ot wishing to be bound by theory, it is proposed that, either on the basis of substrate preference for procollagen II in vivo or on the basis of higher expression in cartilage than ADAMTS-2, ADAMTS-3 is the principal collagen II N-propeptidase in vivo. This is supported by data or observations, including:there is processing of procollagen II despite a null mutation in ADAMTS2; there is roughly equivalent processing of collagen II in transfected RCS-LTC cells by ADAMTS-2 or ADAMTS-3; there are greater than four-fold higher levels of ADAMTS3 than ADAMTS2 MRNA in human cartilage.

There may be other enzymes that contribute substantially to collagen processing in tissues other than skin. To this end, the existence a new member of the PCNP subfamily (ADAMTS-14) was recently identified. Its role in procollagen processing has not yet been studied.

It is proposed herein that ADAMTS-2 and ADAMTS-3 both process procollagen II, but ADAMTS-3 is likely to be more physiologically relevant in this context, possibly due to cartilage-preferred expression. Our data provide insight into the sparing of cartilage and, perhaps, into the relative sparing of some procollagen I-containing tissues in dermatosparaxis. Since it has been shown that ADAMTS-3 will process procollagen II, it is also likely that it will process procollagen I. This is supported by the fact that the related gene family member ADAMTS2 can process both procollagen I and procollagen II. The data presented herein illustrate that ADAMTS-2 and ADAMTS-3 are roughly equivalent in terms of procollagen II processing. Finally, the amino acid cleavage sequence in procollagen I and procollagen II are identical.

Fibrosis and scarring are conditions in which excess fibrous tissue (comprising mainly collagen) is produced following injury, inflammation or attempted repair as for example after surgical incision. In such conditions, the stability and structure of the excess collagen can be rendered inferior and thus less disruptive by preventing the action of ADAMTS-3. Since removal of the N-propeptide is a requirement for formation of collagen fibers and development of an organized collagenous matrix, inhibition of ADAMTS-3 by any means constitutes a potential interference with the fibrosis process and makes ADAMTS-3 a disease or drug target. There are also applications where it may be necessary to introduce or elevate levels of ADAMTS-3. In reconstructing tissues in the art known as tissue engineering, as for example of cartilage, it will be essential to have completely processed procollagen II for optimal organization and function of collagen II. In this case, ADAMTS-3 may be introduced into the tissue engineered product by a variety of means to provide tissue engineered cartilage of optimal function.

While this invention has been described with an emphasis upon preferred embodiments, it will be obvious to those of ordinary skill in the art that variations of the preferred compounds and methods may be used and that it is intended that the invention may be practiced otherwise than as specifically described herein.

What is claimed is:

1. An isolated polynucleotide comprising a nucleic acid sequence encoding an amino acid sequence which is at least 95% identical to SEQ ID NO: 1.

2. The isolated polynucleotide of claim 1, wherein said amino acid sequence comprises SEQ ID NO: 1.

3. The isolated polynucleotide of claim 1, wherein said nucleic acid sequence encodes a metalloprotease.

4. The isolated polynucleotide of claim 1, wherein said polynucleotide comprises SEQ ID NO: 2.

5. An isolated polynucleotide which hybridizes under stringent conditions to a nucleic acid molecule comprising SEQ ID NO: 1 or to a sequence which is complementary to nucleotides of SEQ ID NO: 1.

6. An isolated polynucleotide comprision a sequence which is complementary to the protein encoding sequence of the polynucleotide of claim 1.

7. An expression vector comprising a polynucleotide of claim 1.

8. A host cell transformed or transferred with an expression vector of claim 7.

9. A method for producing ADAMTS-3 protein, said method comprising the steps of
(a) culturing a host cell of claim 8 under conditions suitable for expression of an ADAMTS-3 protein; and
(b) recovering said ADAMTS-3 protein from the host cell culture.

10. The isolated polynucleotide of claim 1 wherein said amino acid sequence is at least 97% identical to SEQ ID NO: 1.

11. The isolated polynucleotide of claim 1 wherein said amino acid sequence which is at least 99% identical to SEQ ID NO: 1.

12. A substantially purfied procollagen peptidase comprised of a peptide selected from the group consisting of functional equivalents of ADAMTS-3, variants of ADAMTS-3, analogs of ADAMTS-3 and fragments of ADAMTS-3.

13. The substantially purified procollagen peptidase of claim 12, wherein said procollagen peptidase is a procollagen I/II N-propeptidase.

14. The substantially purified procollagen peptidase of claim 12 wherein said peptidase is a human peptidase.

15. The substantially purified procollagen peptidase of claim 12 wherein said peptidase is a bovine peptidase.

16. The substantially purified procollagen peptidase of claim 12 wherein said procollagen peptadse is capable of processing procollagens.

17. An isolated polynucleotide comprising a nucleic acid sequence which is at least 95% identical to SEQ ID NO: 2.

18. The isolated polynucleotide of claim 17, said polynucleotide encoding for a protein which processes procollagen II.

19. An isolated polynucleotide comprising a nucleic acid sequence which is at least 95% identical to SEQ ID NO: 4.

20. The isolated polynucleotide of claim 19, said polynucleotide encoding for a protein which processes procollagen II.


SEQUENCE LISTING (4 sequences)

SEQ ID NO: 1 - protein, 1205 aa, Homo sapiens
MVLLSLWLIAAALVEVRTSADGQAGNEEMVQIDLPIKRYREYELVTPVSTNLEGRYLSHTLSASHKKRSARDVSSNPEQLFFNITAFGKDFHLRLKPNTQLVAPGAVVEWHETSLVPGNITDPINNHQPGSATYRIRKTEPLQTNCAYVGDIVDIPGTSVAISNCDGLAGMIKSDNEEYFIEPLERGKQMEEEKGRIHVVYKRSAVEQAPIDMSKDFHYRESDLEGLDDLGTVYGNIHQQLNETMRRRRHAGENDYNIEVLLGVDDSVVRFHGKEHVQNYLLTLMNIVNEIYHDESLGVHINVVLVRMIMLGYAKSISLIERGNPSRSLENVCRWASQQQRSDLNHSEHHDHAIFLTRQDFGPAGMQGYAPVTGMCHPVRSCTLNHEDGFSSAFVVAHETGHVLGMEHDGQGNRCGDETAMGSVMAPLVQAAFHRYHWSRCSGQELKRYIHSYDCLLDDPFDHDWPKLPELPGINYSMDEQCRFDFGVGYKMCTAFRTFDPCKQLWCSHPDNPYFCKTKKGPPLDGTECAAGKWCYKGHCMWKNANQQKQDGNWGSWTKFGSCSRTCGTGVRFRTRQCNNPMPINGGQDCPGVNFEYQLCNTEECQKHFEDFRAQQCQQRNSHFEYQNTKHHWLPYEHPDPKKRCHLYCQSKETGDVAYMKQLVHDGTHCSYKDPYSICVRGECVKVGCDKEIGSNKVEDKCGVCGGDNSHCRTVKGTFTRTPRKLGYLKMFDIPPGARHVLIQEDEASPHILAIKNQATGHYILNGKGEEAKSRTFIDLGVEWDYNIEDDIESLHTDGPLHDPVIVLIIPQENDTRSSLTYKYIIHEDSVPTINSNNVIQEELDTFEWALKSWSQVSKPCGGGFQYTKYGCRRKSDNKMVHRSFCEANKKPKPIRRMCNIQECTHPLWVAEEWEHCTKTCGSSGYQLRTVRCLQPLLDGTNRSVHSKYCMGDRPESRRPCNRVPCPAQWKTGPWSECSVTCGEGTEVRQVLCRAGDHCDGEKPESVRACQLPPCNDEPCLGDKSIFCQMEVLARYCSIPGYNKLCCESCSKRSSTLPPPYLLEAAETHDDVISNPSDLPRSLVMPTSLVPYHSETPAKKMSLSSISSVGGPNAYAAFRPNSKPDGANLRQRSAQQAGSKTVRLVTVPSSPPTKRVHLSSASQMAAASFFAASDSIGASSQARTSKKDGKIIDNRRPTRSSTLER

SEQ ID NO: 2 - DNA, 5822 bp, Homo sapiens
gctttgcccagtagttggaaagtgaactcgactcgtgatggttctcctgtcactttggttgatagcagccgctctggtagaggttaggacttcagctgatggacaagctggtaatgaagaaatggtgcaaatagatttaccaataaagagatatagagagtatgagctggtgactccagtcagcacaaatctagaaggacgctatctctcccatactctttctgcgagtcacaaaaagaggtcagcgagggacgtgtcttccaaccctgagcagttgttctttaacatcacggcatttggaaaagattttcatctgcgactaaagcccaacactcaactagtagctcctggggctgttgtggagtggcatgagacatctctggtgcctgggaatataaccgatcccattaacaaccatcaaccaggaagtgctacgtatagaatccggaaaacagagcctttgcagactaactgtgcttatgttggtgacatcgtggacattccaggaacctctgttgccatcagcaactgtgatggtctggctggaatgataaaaagtgataatgaagagtatttcattgaacccttggaaagaggtaaacagatggaggaagaaaaaggaaggattcatgttgtctacaagagatcagctgtagaacaggctcccatagacatgtccaaagacttccactacagagagtcggacctggaaggccttgatgatctaggtactgtttatggcaacatccaccagcagctgaatgaaacaatgagacgccgcagacacgcgggagaaaacgattacaatatcgaggtactgctgggagtggatgactctgtggtccgtttccatggcaaagagcacgtccaaaactacctcctgaccctaatgaacattgtgaatgaaatttaccatgatgagtccctcggagtgcatataaatgtggtcctggtgcgcatgataatgctgggatatgcaaagtccatcagcctcatagaaaggggaaacccatccagaagcttggagaatgtgtgtcgctgggcgtcccaacagcaaagatctgatctcaaccactctgaacaccatgaccatgcaatttttttaaccaggcaagactttggacctgctggaatgcaaggatatgctccagtcaccggcatgtgtcatccagtgagaagttgtaccctgaatcatgaggatggtttttcatctgcttttgtagtagcccatgaaacgggccatgtgttgggaatggagcatgatggacaaggcaacaggtgtggtgatgagactgctatgggaagtgtcatggctcccttggtacaagcagcattccatcgttaccactggtcccgatgcagtggtcaagaactgaaaagatatatccattcctatgactgtctccttgatgacccttttgatcatgattggcctaaactcccagaacttcctggaatcaattattctatggatgagcaatgtcgttttgattttggtgttggctataaaatgtgcaccgcgttccgaacctttgacccatgtaaacagctgtggtgtagccatcctgataatccctacttttgtaagactaaaaagggacctccacttgatgggactgaatgtgctgctggaaaatggtgctataagggtcattgcatgtggaagaatgctaatcagcaaaaacaagatggcaattgggggtcatggactaaatttggctcctgttctcggacatgtggaactggtgttcgtttcagaacacgccagtgcaataatcccatgcccatcaatggtggtcaggattgtcctggtgttaattttgagtaccagctttgtaacacagaagaatgccaaaaacactttgaggacttcagagcacagcagtgtcagcagcgaaactcccactttgaataccagaataccaaacaccactggttgccatatgaacatcctgaccccaagaaaagatgccacctttactgtcagtccaaggagactggagatgttgcttacatgaaacaactggtgcatgatggaacgcactgttcttacaaagatccatatagcatatgtgtgcgaggagagtgtgtgaaagtgggctgtgataaagaaattggttctaataaggttgaggataagtgtggtgtctgtggaggagataattcccactgccgaaccgtgaaggggacatttaccagaactcccaggaagcttgggtaccttaagatgtttgatataccccctggggctagacatgtgttaatccaagaagacgaggcttctcctcatattcttgctattaagaaccaggctacaggccattatattttaaatggcaaaggggaggaagccaagtcgcggaccttcatagatcttggtgtggagtgggattataacattgaagatgacattgaaagtcttcacaccgatggacctttacatgatcctgttattgttttgattatacctcaagaaaatgatacccgctctagcctgacatataagtacatcatccatgaagactctgtacctacaatcaacagcaacaatgtcatccaggaagaattagatacttttgagtgggctttgaagagctggtctcaggtttccaaaccctgtggtggaggtttccagtacactaaatatggatgccgtaggaaaagtgataataaaatggtccatcgcagcttctgtgaggccaacaaaaagccgaaacctattagacgaatgtgcaatattcaagagtgtacacatccactctgggtagcagaagaatgggaacactgcaccaaaacctgtggaagttctggctatcagcttcgcactgtacgctgccttcagccactccttgatggcaccaaccgctctgtgcacagcaaatactgcatgggtgaccgtcccgagagccgccggccctgtaacagagtgccctgccctgcacagtggaaaacaggaccctggagtgagtgttcagtgacctgcggtgaaggaacggaggtgaggcaggtcctctgcagggctggggaccactgtgatggtgaaaagcctgagtcggtcagagcctgtcaactgcctccttgtaatgatgaaccatgtttgggagacaagtccatattctgtcaaatggaagtgttggcacgatactgctccataccaggttataacaagttatgttgtgagtcctgcagcaagcgcagtagcaccctgccaccaccataccttctagaagctgctgaaactcatgatgatgtcatctctaaccctagtgacctccctagatctctagtgatgcctacatctttggttccttatcattcagagacccctgcaaagaagatgtctttgagtagcatctcttcagtgggaggtccaaatgcatatgctgctttcaggccaaacagtaaacctgatggtgctaatttacgccagaggagtgctcagcaagcaggaagtaagactgtgagactggtcaccgtaccatcctccccacccaccaagagggtccacctcagttcagcttcacaaatggctgctgcttccttctttgcagccagtgattcaataggtgcttcttctcaggcaagaacctcaaagaaagatggaaagatcattgacaacagacgtccgacaagatcatccaccttagaaagatgagaaagtgaaccaaaaaggctagaaaccagaggaaaacctggacaacctctctcttcccatggtgcatatgcttgtttaaagtggaaatctctatagatcgtcagctcattttatctgtaattggaagaacagaaagtgctggctcactttctagttgctttcatcctccttttgttctgcattgactcatttaccagaattcattggaagaaatcaccaaagattattacaaaagaaaaatatgttgctaagattgtgttggtcgctctctgaagcagaaaagggactggaaccaattgtgcatatcagctgactttttgtttgttttagaaaagttacagtaaaaattaaaaagagataccaatggtttacactttaacaagaaattttggatatggaacaaagaattcttagacttgtattcctatttatctatattagaaatattgtatgagcaaatttgcagctgttgtgtaaatactgtatattgcaaaaatcagtattattttaagagatgtgttctcaaatgattgtttactatattacatttctggatgttctaggtgcctgtcgttgagtattgccttgtttgacattctataggttaattttcaaagcagagtattacaaaagagaagttagaattacagctactgacaatataaagggttttgttgaatcaacaatgtgatacgtaaattatagaaaaagaaaagaaacacaaaagctatagatatacagatatcagcttacctattgccttctatacttataatttaaaggattggtgtcttagtacacttgtggtcacagggatcaacgaatagtaaataatgaactcgtgcaagacaaaactgaaaccctctttccaggacctcagtaggcaccgttgaggtgtcctttgtttttgtgtgtgtgtgttcttttttaattttcgcattgttgacagatacaaacagttatactcaatgtactgtaataatcgcaaaggaaaaagttttgggataacttatttgtatgttggtagctgagaaaaatatcatcagtctagaattgatatttgagtatagtagagctttggggctttgaaggcaggttcaagaaagcatatgtcgatggttgagatatttattttccatatggttcatgttcaaatgttcacaaccacaatgcatctgactgcaataatgtgctaataatttatgtcagtagtcaccttgctcacagcaaagccagaaatgctctctccagggagtagatgtaaagtacttgtacatagaattcagaactgaagatatttattaaaagttgatttttttttcttgatagtatttttatgtactaaatatttacactaatatcaattacatattttggtaaactagagagacataattagagatgcatgctttgttctgtgcatagagacctttaagcaaactactacagccaactcaaaagctaaaactgaacaaatttgatgttatgcaaacatcttgcatttttagtagttgatattaagttgatgacttgtttcccttcaaggaaacattaaattgtatggactcagctagctgttcaatgaaattgtgaattagaaacatttttaaaagtttttgaaagagataagtgcatcatgaattacatgtacatgagaggagatagtgatatcagcataatgattttgaggtcagtacctgagctgtctaaaaatatattatacaaactaaaatgtagatgaattaacctctcaaagcacagaatgtgcaagaacttttgcattttaatcgttgtaaactaacagcttaaactattgactctatacctctaaagaattgctgctactttgtgcaagaactttgaaggtcaaattaggcaaattccagatagtaaaacaatccctaagccttaagtcttttttttttcctaaaaattcccatagaataaaattctctctagtttacttgtgtgtgcatacatctcatccacaggggaagataaagatggtcacacaaacagtttccataaagatgtacatattcattatacttctgacctttgggctttcttttctactaagctaaaaattcctttttatcaaagtgtacactactgatgctgtttgttgtactgagagcacgtaccaataaaaatgttaacaaaatat

SEQ ID NO: 3 - protein, 227 aa, Homo sapiens
MVLLSLWLIAAALVEVRTSADGQAGNEEMVQIDLPIKRYREYELVTPVSTNLEGRYLSHTLSASHKKRSARDVSSNPEQLFFNITAFGKDFHLRLKPNTQLVAPGAVVEWHETSLVPGNITDPINNHQPGSATYRIRKTEPLQTNCAYVGDIVDIPGTSVAISNCDGLAGMIKSDNEEYFIEPLERGKQMEEEKGRIHVVYKRSAVEQAPIDMSKDFHYRESDLEGL

SEQ ID NO: 4 - DNA, 720 bp, Homo sapiens
gctttgcccagtagttggaaagtgaactcgactcgtgatggttctcctgtcactttggttgatagcagccgctctggtagaggttaggacttcagctgatggacaagctggtaatgaagaaatggtgcaaatagatttaccaataaagagatatagagagtatgagctggtgactccagtcagcacaaatctagaaggacgctatctctcccatactctttctgcgagtcacaaaaagaggtcagcgagggacgtgtcttccaaccctgagcagttgttctttaacatcacggcatttggaaaagattttcatctgcgactaaagcccaacactcaactagtagctcctggggctgttgtggagtggcatgagacatctctggtgcctgggaatataaccgatcccattaacaaccatcaaccaggaagtgctacgtatagaatccggaaaacagagcctttgcagactaactgtgcttatgttggtgacatcgtggacattccaggaacctctgttgccatcagcaactgtgatggtctggctggaatgataaaaagtgataatgaagagtatttcattgaacccttggaaagaggtaaacagatggaggaagaaaaaggaaggattcatgttgtctacaagagatcagctgtagaacaggctcccatagacatgtccaaagacttccactacagagagtcggacctggaaggccttga